(19) United States
(12) Patent Application Publication
(10) Pub. No.: US 2016/0256504 A1
(43) Pub. Date: Sep. 8, 2016

(54) ANTIHYPERTENSIVE COMPOSITION CONTAINING SHIITAKE MUSHROOM [LENTINUS EDODES] MYCELIUM CULTURE AND NATTO BACTERIA [BACILLUS SUBTILIS NATTO] CULTURE

(71) Applicant: HK Biotech Co., LTD., Jinju-si, Gyeongsangnam-do (KR)

(72) Inventor: Jeong Ok KIM, Jinju-si, Gyeongsangnam-do (KR)

(73) Assignee: HK Biotech Co., LTD., Jinju-si, Gyeongsangnam-do (KR)

(21) Appl. No.: 14/406,156

(22) PCT Filed: May 21, 2014
(86) PCT No.: PCT/KR2014/004515

§ 371 Date: Dec. 5, 2014

(30) Foreign Application Priority Data
May 20, 2014 (KR) 10-2014-0060425

(51) Int. Cl. A61K 36/07; A01G 1/04; A61K 35/742

(52) U.S. Cl. CPC A61K 36/07; A61K 35/742; A23L 1/3014; A23L 1/28; A01G 1/04; A23V 2002/00

(57) ABSTRACT

The present disclosure proposes an antihypertensive component and a method for increasing the content of EA in the shiitake mushroom mycelium culture. The antihypertensive component according to an exemplary embodiment of the present disclosure contains both of shiitake mushroom mycelium culture and natto bacteria culture, and has an effect of enhancing antihypertensive efficacy by thrombolysis ability of the natto bacteria culture, even though the EA content may be lowered by combination of the natto bacteria culture.

### BACKGROUND

1. Field of the Disclosure

The present disclosure relates to a composition having antihypertensive efficacy. More specifically, the present disclosure relates to a composition to enhance antihypertensive efficacy by combination of cultures.

2. Discussion of the Related Art

The hypertension (more than 140/90 mmHg) is coming to the fore as a big problem in an aging society. According to the health statistics published in 2010 by Korean Ministry of Health & Welfare, the hypertension prevalence is proportional to the age. Thus, along with increasing population of over-sixties, the hypertension is on the rise as a serious problem, not only in Korea, but also throughout the whole world.

It is reported that 12.6% of males in thirties, 1.6% of females in thirties, 50.1% of males in seventies, 68.2% of females in seventies; and more than 50% of over-sixties populations have hypertension symptoms. 67 million U.S. adults (one out of every three adults) are classified as having hypertensions, and a half of them (36 million people) are exposed to the risk of myocardial infarction, because they are not receiving effective treatment and management.

The general hypertension therapeutic purpose ‘new medicine’ occupies an important position. However, it has a problem of causing a side effect when taking for a long period of time. Therefore, it is required to develop a natural material of new hypertension therapeutic medicine which does not cause any side effect even when taking for a long period of time.

Among the natural materials, an ingredient called ‘EA (eritadenine)’, which is contained in Shiitake mushroom (Lentinus edodes) mycelium, is reported to have an effect of keeping the heart healthy and decreasing blood cholesterol concentration. That is, when injecting EA into rats hyperhomocysteinemia induced by guanidinoacetic acid injection, it is reported that blood homocysteine, which is a hypercholesterolemia-related biomarker, of the rat is reduced (Shin-ichiro, Fukada et al., J. Nutr. 136:2797˜2802, 2006). This is the effect of when daily administering 9 g of shiitake mushroom mycelium of which EA content is 3.6 mg/100 g to 6.3 mg/100 g into a rat.

In addition, the functional health food material is suitable to prevent chronic diseases because it does not cause side effects. However, it is required to be ingested in a large amount to show equal effect to that of hypertension therapeutic medicine. In addition, in order to develop shiitake mushrooms or shiitake mushroom mycelium culture as a hypertension therapeutic natural material for new medicine, the content of EA, which is the active constituent contained in them, is required to be increased.

In general, a small amount of EA is contained in shiitake mushrooms or shiitake mushroom mycelium. While the shiitake mushroom mycelium culture of HK Biotech contains 0.4 mg/100 g of EA in dry matter and the shiitake mushroom fruit body contains 0.3 mg/100 g of EA in dry matter, the other edible mushrooms hardly contain any EA. It is reported that European mushroom contains 0.07 mg/100 g of EA in dry matter, while shimeji mushroom, black mushroom, winter mushroom, and Pholiota nameko have not been reported to contain any EA.

### SUMMARY OF THE DISCLOSURE

As a solution to the above mentioned problems, the present disclosure provides a composition of which antihypertensive effect is excellent, containing natural product or its culture a main ingredient.

In addition, the present disclosure provides a method to increase the content of EA which is known to be contained in shiitake mushroom mycelium culture and to have an effect of deceasing cholesterol concentration.

In order to accomplish the aforementioned purpose, in one general aspect of the present disclosure, there is provided an antihypertensive composition, the antihypertensive composition comprising shiitake mushroom mycelia and natto bacteria culture.

In some exemplary embodiments of the present disclosure, the shiitake mushroom mycelium culture may contain mycelia obtained by culturing shiitake mushroom (Lentinus edodes) strain, and the natto bacteria culture is obtained by culturing natto bacteria (Bacillus subtilis natto).

In some exemplary embodiments of the present disclosure, the ratio of the shiitake mushroom mycelium culture and the natto bacteria culture may be in a weight ratio of 99˜95:1˜5, respectively.

In some exemplary embodiments of the present disclosure, the shiitake mushroom mycelium culture may be obtained by culturing shiitake mushroom (Lentinus edodes) strain in a medium to which adenine or ribose or both are added purposely.

In some exemplary embodiments of the present disclosure, the shiitake mushroom mycelium culture may be obtained by culturing shiitake mushroom (Lentinus edodes) strain in a medium containing food additive or yeast extract containing adenine and ribose.

In some exemplary embodiments of the present disclosure, the shiitake mushroom mycelium culture may be obtained by culturing mycelia, obtained by culturing shiitake mushroom (Lentinus edodes) strain, in a medium in which adenine or ribose or both are naturally present.

In some exemplary embodiments of the present disclosure, the medium may contain the adenine and the ribose in an amount of 0.1˜0.5 weight % of a total weight of the medium.

In another general aspect of the present disclosure, there may be provided a hypertension therapeutic or preventive pharmaceutical composition, comprising the antihypertensive composition.

In still another general aspect of the present disclosure, there may be provided an antihypertensive health food, comprising the antihypertensive composition.

In still another general aspect of the present disclosure, there is provided a method for increasing EA content in shiitake mushroom mycelium culture, comprising cultivating shiitake mushroom (Lentinus edodes) strain in a medium containing adenine and ribose.

In some exemplary embodiments of the present disclosure, the medium may contain each of the adenine and the ribose in an amount of 0.1˜0.5 weight % of a total weight of the medium.

Other details of exemplary embodiments of the present disclosure are contained in the figures and the detailed description.

Such composition according to an exemplary embodiment of the present disclosure contains shiitake mushroom mycelium culture and natto bacteria culture as well, and has an effect of enhancing antihypertensive efficacy by thrombolysis ability of the natto bacteria culture, even though the EA content rate may be lowered by combination of the natto bacteria culture.

In addition, the present disclosure has an effect of prominently increasing the content of EA in the shiitake mushroom mycelium culture, by proceeding through a step to culture the shiitake mushroom strain in a medium containing adenine and ribose.

### BRIEF DESCRIPTION OF THE DRAWINGS

FIG. 1 is a graph illustrating a systolic blood pressure (SBP) drop effect of a mixture of shiitake mushroom mycelium culture and natto bacteria culture on L-N-Nitro-L- arginine methyl ester hydrochloride (NAME)-induced hypertensive rats according to an exemplary embodiment of the present disclosure.

FIG. 2 is a graph illustrating a diastolic blood pressure (DBP) drop effect of a mixture of shiitake mushroom mycelium culture and natto bacteria culture on L-N-Nitro-L- arginine methyl ester hydrochloride (NAME)-induced hypertensive rats according to an exemplary embodiment of the present disclosure.

FIG. 3 is a graph illustrating a mean blood pressure (MBP) drop effect of a mixture of shiitake mushroom mycelium culture and natto bacteria culture on L-N-Nitro-L- arginine methyl ester hydrochloride (NAME)-induced hypertensive rats according to an exemplary embodiment of the present disclosure.

FIG. 4 is a graph illustrating a systolic blood pressure (SBP) drop effect of a mixture of shiitake mushroom mycelium culture and natto bacteria culture by short-term treatments on spontaneously hypertensive rat (SHR)s according to an exemplary embodiment of the present disclosure.

FIG. 5 is a graph illustrating a diastolic blood pressure (DBP) drop effect of a mixture of shiitake mushroom mycelium culture and natto bacteria culture by short-term treatments on spontaneously hypertensive rat (SHR)s according to an exemplary embodiment of the present disclosure.

FIG. 6 is a graph illustrating a mean blood pressure (MBP) drop effect of a mixture of shiitake mushroom mycelium culture and natto bacteria culture by short-term treatments on spontaneously hypertensive rat (SHR)s according to an exemplary embodiment of the present disclosure.

FIG. 7 is a graph illustrating a systolic blood pressure (SBP) drop effect of a mixture of shiitake mushroom mycelium culture and natto bacteria culture by long-term treatments on spontaneously hypertensive rat (SHR)s according to an exemplary embodiment of the present disclosure.

FIG. 8 is a graph illustrating a diastolic blood pressure (DBP) drop effect of a mixture of shiitake mushroom mycelium culture and natto bacteria culture by long-term treatments on spontaneously hypertensive rat (SHR)s according to an exemplary embodiment of the present disclosure.

FIG. 9 is a graph illustrating a mean blood pressure (MBP) drop effect of a mixture of shiitake mushroom mycelium culture and natto bacteria culture by long-term treatments on spontaneously hypertensive rat (SHR)s according to an exemplary embodiment of the present disclosure.

### DETAILED DESCRIPTION

Various exemplary embodiments will be described more fully hereinafter with reference to the accompanying drawings, in which some exemplary embodiments are shown. The present inventive concept may, however, be embodied in many different forms and should not be construed as limited to the exemplary embodiments set forth herein. Rather, the described aspect is intended to embrace all such alterations, modifications, variations, and equivalents that fall within the scope and novel idea of the present disclosure. When it is determined that a detailed description about known function or structure relating to the present disclosure may evade the main point of the present disclosure, the detailed description may be omitted.

The terms mentioned herein are used merely for description of a particular exemplary embodiment. Thus, they are not intended to limit the scope of the present disclosure. The singular expression contains numeral expression thereof unless it has a clearly different meaning in the context. In the present disclosure, the terms such as “include”, “contain”, or “have” are state that there may be in existence of features, numbers, steps, functions, elements, components described herein, or compositions thereof. Therefore, they shall not be understood as to exclude the possibility of existence or addition of one or more other features, numbers, steps, functions, elements, components described herein, or compositions thereof.

The terms such as “first” or “second” may be used for description of various elements. However, the elements shall not be limited by such the terms. The terms are used merely to distinguish a particular element from another element.

The present disclosure provides an antihypertensive composition containing shiitake mushroom mycelium culture and natto bacteria culture. That is, the present disclosure relates to a composition which is able to enhance antihypertensive ability remarkably, by mixing shiitake mushroom mycelium culture with natto bacteria culture.

Researchers of the present disclosure ascertained that there occur frequently the cases where the blood pressures of the hypertensive patients who have ingested shiitake mushroom mycelium and natto bacteria culture simultaneously are normalized. Afterwards, the researcher invented a composition in which shiitake mushroom mycelium culture with natto bacteria culture, as an antihypertensive natural composition.

Such the composition is a natural composition which has similar or enhanced antihypertensive efficacy compare to the conventional medicines, and at the same time, has no side effect which are found in the conventional new medicines.

Here, the shiitake mushroom mycelium culture may contain mycelia obtained by cultivating shiitake mushroom (Lentinus edodes) strain, and the natto bacteria culture may be obtained by cultivating natto bacteria (Bacillus subtilis natto).

In general, the EA contained in the culture of shiitake mushroom fruit body or mycelia is known to keep the heart healthy, to reduce the concentration of blood cholesterol, and to lower the content of blood homocysteine. However, the EA contained in the conventional culture of shiitake mushroom fruit body or mycelia suffers from a problem that its content is very insignificant.

Apart from the foregoing, in general, when soybeans are naturally ripened under a temperature of 30-40° C., ferment bacteria generating transparent slime. These bacteria are what we call Bacillus natto bacteria (Bacillus subtilis natto). Seven species of Bacillus subtilis natto are registered at ATCC, and other various strains are separated from the natural ripening to be reported. For a long time, these bacteria fermented soybeans have been used as a food in Korea (referred to as “cheonggukjang”) and Japan (referred to as “natto”). Recently, they are also used in western countries such as the U.S. as a food or a food additive, and registered at FDA as a GRAS (Generally recognized as safe) material. Among various bioactive functions of such natto bacteria fermented materials, the thrombolysis function is already discovered by numerous research materials.

The generation of thrombus in blood vessels debases the blood liquidity. This turns out as symptoms of cardiovascular diseases such as hypertension, cerebrovascular accident, etc. In a living matter, the blood always keeps the balance between coagulation and dissolution. Thus, while the blood is circulating normally, the generated thrombus is dissolved immediately [Arbige and Pitcher, 1989]. The thrombus is generated whereby fibrinogen in the blood is transformed into fibrin by thrombin when the living matter is wounded. When a thrombus is generated in the vein, it leads to an insufficient blood circulation causing an edema or an infection. However, when a thrombus is generated in the artery, it leads to an ischemia or an infarct, causing a hypertension and cardiovascular diseases such as arteriosclerosis, myocardial infarction, cerebrovascular accident, and pulmonary thromboembolism [Grande et al. 1990; Ruggeri and Zimmerman 1985; Sherman and Hart 1986; Shin et al. 2007].

Nattokinase is a kind of microbial serine protease. When ingested by oral administration, it is absorbed into the body and acting as a plasminogen activator to transform plasminogen into plasmin. Such the plasmin dissolves fibrin of the coagulated blood to have fibrinolytic activity [Kim, 2002; Sumi 1990; Yang 2000; Yong et al. 2005].

The nattokinase does not only decompose doss-linked fibrin directly in vitro, but also accelerates generation of tPA, and regulates thrombolysis ability by the relative proportion of the tPA, and reinforces the thrombolysis through inactivity and decomposition of the plasminogen activator inhibitor-1 (PAI-1) which is the first inhibitor of fibrin clot lysis [Fujita et al. 1995; Urano et al. 2001].

Likewise, the nattokinase is known to have thrombolysis ability. However, in a general natto culture (NT) does not contain the EA. Therefore, when generally mixing shiitake mushroom mycelium culture (LE) and natto culture (NT), it is commonly expected that the mixture contains less EA content and thus the antihypertensive efficacy will be reduced.

However, as confirmed in the following experimental examples, the mixture of shiitake mushroom mycelium culture and natto culture according to an exemplary embodiment of the present disclosure has remarkably enhanced antihypertensive ability, compared to each of the shiitake mushroom mycelium culture and the natto culture, respectively. Thus, it is considered that: although the EA content is decreased with the increasing of the natto culture content in the mixture, the decrement is not significant; rather, the antihypertensive efficacy is enhanced whereby the natto culture is contained in the mixture to promote the blood circulation with the thrombolysis ability of the natto culture.

According to an exemplary embodiment of the present disclosure, in order to enhance the antihypertensive efficacy of the shiitake mushroom mycelium culture, the natto bacteria culture which has the thrombolysis ability is mixed with the shiitake mushroom mycelium culture. Through this process, it is confirmed that remarkably enhanced antihypertensive efficacy may be obtained compared to the conventions. For example, by preparing each of the shiitake mushroom mycelium culture and the natto bacteria culture and mixing them, the synergy effect between the two ingredients may be enhanced.

Here, the shiitake mushroom mycelium culture and the natto bacteria culture may be contained in a weight ratio of 99˜95:1˜5. When the content of the natto bacteria culture is lowered below the aforementioned range, the thrombolysis activity may be decreased. When the content of the natto bacteria is higher than the range, the EA content in the composition or the antihypertensive efficacy may be decreased.

Meanwhile, according to another exemplary embodiment of the present disclosure, in order to increase the content of EA in the shiitake mushroom mycelium culture, the shiitake mushroom mycelium culture may be obtained by cultivating shiitake mushroom strain in a medium containing adenine and ribose.

That is, when cultivating the shiitake mushroom strain, the shiitake mushroom strain is cultured in a liquid or solid medium which is further containing the precursors of the EA such as adenine, ribose, etc. Through this process, the turnover rate of adenine and ribose to EA may be increased, and the content of the EA may be increased.

For example, according to some exemplary embodiments of the present disclosure, the shiitake mushroom mycelium culture in which EA content is increased may be obtained by cultivating shiitake mushroom strain in a medium containing adenine and ribose. In addition, the shiitake mushroom mycelium culture may be obtained by cultivating shiitake mushroom strain in a medium containing food additive or yeast extract containing adenine and ribose. That is, when using food additive or yeast extract containing adenine instead of the adenine itself, it may be more comfortable and efficient to produce as a health food composition. In addition, the shiitake mushroom mycelium culture in which EA content is increased may be obtained by cultivating mycelia obtained by cultivating shiitake mushroom strain, in a medium containing adenine and ribose.

Here, the medium containing the medium containing adenine and ribose may contain each of the adenine and the ribose in an amount of 0.1˜0.5 weight % of a total weight of the medium, respectively. When the content of the adenine and the ribose is lowered below the aforementioned range, the total increment of the EA content may be insignificant. When the content of the adenine and the ribose is higher than the range, the turnover rate to the EA and the EA content itself may be decreased.

According to some exemplary embodiments of the present disclosure, the method for increasing content of the EA which is the antihypertensive index ingredient in the shiitake mushroom mycelium culture may further contain a cultivation method, an extraction method, and a concentration method. The cultivation method may be a changed composition of the medium in which the mycelium is to be cultured. Afterwards, the extraction and the concentration method may be increasing the content of the EA which is the antihypertensive index ingredient up to above 20 mg/g to be industrially available.

Along with this, according to another exemplary embodiment of the present disclosure, there may be provided a hypertension therapeutic or preventive pharmaceutical composition containing the antihypertensive composition.

According to an exemplary embodiment of the present disclosure, the composition containing shiitake mushroom mycelium and natto bacteria culture has an excellent antihypertensive efficacy to use for hypertension treatment or prevention.

Herein, the “prevention” means all kinds of actions for suppressing formation of a disease or delaying occurrence of the disease by administering a composition. And the “treatment” means all kinds of actions for improving or making beneficial changes to symptoms of the disease by administering the composition.

The composition containing the antihypertensive composition according to an exemplary embodiment of the present disclosure may be used as a pharmacological composition by further containing a pharmacologically acceptable carrier, and may be manufactured as a medicine with the carrier. In addition, the composition according to an exemplary embodiment of the present disclosure may be used as a single medicine, or may be used as a complex medicine along with other active constituents which may be able to enhance the efficacy of the medicine.

Furthermore, according to another exemplary embodiment of the present disclosure, there may be provided an antihypertensive health food containing the antihypertensive composition.

The composition according to an exemplary embodiment of the present disclosure may be manufactured as a medicine by further containing sitologically acceptable food additives, and may be used as hypertension therapeutic or preventive health functional food. The health functional food according to an exemplary embodiment of the present disclosure may include the forms of a tablet, a pill, and a liquid medicine.

The composition according to an exemplary embodiment of the present disclosure may be added to the food such as various foodstuffs, beverages, chewing gums, teas, vitamin complexes, and health functional foodstuffs.

The following exemplary embodiments will provide more specific and detailed description for the present disclosure. However, the following exemplary embodiments is not for limiting the scope of the present disclosure as claimed, but only for providing examples of the embodiments of the present disclosure.

### Exemplary Embodiment 1

### Culturing Shiitake Mushroom Mycelia which May Increase the Content of EA

### Exemplary Embodiment 1-1

### Liquid Culture

1) Erlenmeyer Flask (500 ml) Culture

A liquid medium formed of soybean hydrolysate 0.4%, brown sugar 2%, MgSO₄7H₂O 0.05%, and KH₂PO4 0.05% are added to a Erlenmeyer flask (500 ml). Afterwards, the Erlenmeyer flask is high-pressure sterilized (121° C., 30 minutes) and then is cooled to the room temperature. Thereon, the shiitake mushroom (Lentinus edodus) strain cultured on a PDA medium is cut in slices below 7 mm in diameter to be inoculated. Then the inoculated shiitake mushroom strains are cultured in a shaking incubator (130 rpm, 25° C., 8 days).

2) 30 L Liquid Culture

The compositions of the mediums are differentiated by adding additives such as adenine and ribose, as described in the following TABLE 1, to the basic liquid medium formed of soybean hydrolysate 0.4%, brown sugar 2%, MgSO₄7H₂O 0.05%, and KH₂PO4 0.05%. Such the medium (30 L) containing the EA precursor is added to a fermentor (50 L). Afterwards, the culture medium is high-pressure sterilized (121° C., 60 minutes) and then is cooled to the room temperature. Thereon, 1500 ml of the shiitake mushroom mycelium culture prepared by the method of step 1) is inoculated and then cultured for 8 days at the temperature of 25° C.

Afterwards, the content of EA contained in the culture medium is measured by the HPLC method with caffeic acid as an internal standard material. The result is as described in the following TABLE 1.

TABLE 1

| | | EA Content [mg % (mg/100 ml | | | |
|---|---|---|---|---|---|
| | | Culture medium)]¹⁾ | | | |
| | | 0 | 1 | 4 | 8 |
| NO. | Medium Composition | week | week | weeks | weeks |
| 1 | Basic medium + LE mycelia | — | 0.1²⁾ | 0.5 | 0.6 |
| 2 | Basic medium + 0.01% adenine + 0.01% ribose + | — | 1 | 13 | 12 |
| | LE mycelia | | | | (100% |
| | | | | | conversion) |
| 3 | Basic medium + 0.1% adenine + 0.1% ribose + LE | — | 11 | 93 | 102 |
| | mycelia | | | | (100% |
| | | | | | conversion) |
| 4 | Basic medium + 0.5% adenine + 0.5% ribose + LE | — | 36 | 358 | 383 |
| | mycelia | | | | (76.6% |
| | | | | | conversion) |
| 5 | Basic medium + 1.0% adenine + 1.0% ribose + | — | 47 | 319 | 395 |
| | LE mycelia | | | | (30% |
| | | | | | conversion) |
| 6 | Basic medium + 2.0% adenine + 2.0% ribose + | — | 36 | 113 | 137 |
| | LE mycelia | | | | (14% |
| | | | | | conversion) |
| | | | | | rate |
| 7 | Basic medium + 1.0% yeast extract + 1.0% | — | 25 | 134 | 256 |
| | ribose + LE mycelia | | | | |
| 8 | Basic medium + 1.0% Bio heksane³⁾ + 1.0% | — | 33 | 146 | 301 |
| | ribose + LE mycelia | | | | |
| 9 | Basic medium + 1.0% yeast extract + 1.0% Bio | — | 34 | 177 | 297 |
| | heksane + 1.0% ribose + LE mycelia | | | | |

¹⁾mg % (mg/100 g dry matter): around 1%-2% in dry matter
²⁾Standard deviation of 3-time-repetition is within 5%.
³⁾Food additive containing disodium 5′-inosinate in the amount of above 97% and adenine (e.g., Bio heksane ®)

As shown in the above TABLE 1, specimens 3 and 4 have the highest EA content and the highest conversion rate of adenine and ribose. That is, specimen 3 (0.1% treatment), where no residue amount of adenine and ribose is generated, has the most enhanced efficacy. In the order of the total EA generation amount, specimen 4 (0.5% treatment) generates the highest amount of EA. In the medium to which adenine and ribose are added, 100% of specimen 3 (each of adenine and ribose is added by 0.1%, respectively) is converted to the EA after 8-week-culture, and 77% of specimen 4 (each of adenine and ribose is added by 0.5%, respectively) is converted to the EA after 8-week-culture. When the concentration of adenine or ribose is above 1%, it is observed that their conversion rate to the EA was considerably decreased.

In addition, when using food additive or yeast extract containing adenine instead of the adenine itself (specimens 7, 8, and 9), it is observed that relatively high EA content and was obtained.

### Exemplary Embodiment 1-2

### Solid Culture

In this exemplary embodiment, there may be manufactured a medium where the shiitake mushroom mycelia grow favorably and the EA content may be maximized. For this purpose, barley and rice bran in the weight ratio of 9:1, respectively, are prepared as a basic medium. And then, the compositions of the mediums are differentiated by adding additives such as adenine and ribose, as described in the following TABLE 2, to the basic medium. Afterwards, such the medium is sterilized (121° C., 3 hours) and then is cooled to the room temperature. Thereon, the shiitake mushroom mycelium culture cultured by the method of step 1) of Exemplary Embodiment 1 is inoculated (50 ml/kg medium) and then cultured for 8 weeks at the temperature of 25° C. to obtain the solid culture.

Afterwards, the content of EA contained in the culture is measured by the HPLC method (with caffeic acid as an internal standard material). The result is as described in the following TABLE 2.

TABLE 2

| | | EA Content [mg % (mg/100 g | | | |
|---|---|---|---|---|---|
| | | Culture)]¹⁾ | | | |
| | | 0 | 1 | 4 | 8 |
| NO. | Medium Composition | week | week | weeks | weeks |
| 1 | Basic medium + LE mycelia | — | 9²⁾ | 55 | 98 |
| 2 | Basic medium + 0.5% adenine + | — | 134 | 845 | 1,035 |
| | 0.5% ribose + LE mycelia | | | | |
| 3 | Basic medium + 1.0% yeast | — | 51 | 402 | 656 |
| | extract + 1.0% ribose + LE | | | | |
| | mycelia | | | | |
| 4 | Basic medium + 1.0% Bio | — | 43 | 290 | 677 |
| | heksane³⁾ + 1.0% ribose + | | | | |
| | LE mycelia | | | | |
| 5 | Basic medium + 1.0% yeast | — | 53 | 312 | 761 |
| | extract + 1.0% Bio heksane + | | | | |
| | 1.0% ribose + LE mycelia | | | | |

¹⁾mg % (mg/100 g culture): moisture 50%
²⁾Standard deviation of 3-time-repetition is within 5%.
³⁾Food additive containing disodium 5′-inosinate in the amount of above 97% and adenine (e.g., Bio heksane ®)

As shown in the above TABLE 2, specimens 2˜5 have higher EA content and turnover rate than those of specimen 1, in totality. Among the specimens 2˜5, specimen 2 has the highest EA content and conversion rate of adenine and ribose.

### Exemplary Embodiment 2

### Manufacturing a Composition in which Shiitake Mushroom Mycelium Culture and Natto Bacteria Culture are Mixed

### Exemplary Embodiment 2-1

### Manufacturing Dried Powder of Shiitake Mushroom Mycelium Solid Culture

The shiitake mushroom mycelium solid culture manufactured through the exemplary embodiment 1-2 is hot-air-dried at the temperature of 80° C. for 72 hours to be manufactured as dry specimen containing moisture content in the amount of below 4%. Afterwards, the dry specimen is pulverized into powder (100 mesh).

### Exemplary Embodiment 2-2

### Manufacturing Nattokinase Concentrate Powder

At first, the nattokinase concentrate (NKC) is manufactured. That is, using a sterilized liquid medium (121° C., 1 hour) containing defatted soybean powder (5%) and maltose (1%), the natto bacteria (Bacillus subtilis natto) purchased from Korean Culture Center of Microorganism (KCCM) is cultured in a 5 L-fermentor (Kobiotech, Co., Incheon, Korea) for 48 hours. And then, the culture is filtered out and concentrated for its fibrinolytic activity to be above 5,000 U/ml.

Afterwards, the nattokinase concentrate is pulverized into powder. That is, the nattokinase concentrate (NKC) manufactured in the above is mixed with indigestible maltodextrin to be adjusted so that its solid content becomes 35%. And then the mixture is spray-dried (by J-004 of JEIL Machine, Co., Ltd.) to be manufactured as powder.

### Exemplary Embodiment 2-3

### Manufacturing Hot-Water-Extracts in which Shiitake Mushroom Mycelium Culture and Natto Bacteria Culture are Mixed

At first, a mixture composition of shiitake mushroom mycelium culture and natto bacteria culture is manufactured. That is, the dried powder of shiitake mushroom mycelium solid culture (8-week-culture) manufactured through the above exemplary embodiment 2-1 and the nattokinase concentrate powder manufactured through the above exemplary embodiment 2-1 are mixed by the ratio as described in the following TABLE 3.

TABLE 3

| | Spec- | Content ratio of shiitake | Content ratio of natto |
|---|---|---|---|
| Manu- | imen | mushroom mycelium culture | bacteria culture |
| facture | name | (by weight) | (by weight) |
| 1 | NT | 0 | 100 |
| 2 | LE | 100 | 0 |
| 3 | LN1 | 99 | 1 |
| 4 | LN3 | 97 | 3 |
| 5 | LN5 | 95 | 5 |

Afterwards, each of the mixtures (100 g) in the above is mixed with 400 mL of purified water, and then centrifugated (5,000 rpm×30 minutes) for its supernatant to be obtained. The EA content contained in the supernatant is measured. The result is as described in the following TABLE 4.

TABLE 4

| | | Content ratio of | Content ratio of | | | EA |
|---|---|---|---|---|---|---|
| | | shiitake mushroom | natto bacteria | Hot-water- | Solid | content |
| | Specimen | mycelium culture | culture | extracts | content | (mg/100 |
| NO. | name | (by weight) | (by weight) | (ml) | (%) | ml) |
| 1 | NT | 0 | 100 | 350 | 13 | — |
| 2 | LE | 100 | 0 | 350 | 13 | 289 |
| 3 | LN1 | 99 | 1 | 350 | 13 | 283 |
| 4 | LN3 | 97 | 3 | 350 | 13 | 277 |
| 5 | LN5 | 95 | 5 | 350 | 13 | 271 |

As shown in the above TABLE 4, as the natto bacteria culture (NT) increases, the content of EA contained in the mixture gradually decreases.

This is because EA is not contained in the natto bacteria culture (NT). Thus, in general, it may be easily expected that the antihypertensive efficacy of the mixture of shiitake mushroom mycelium culture (LE) and natto bacteria culture (NT) will be reduced, because the mixture contains less content of EA. However, as confirmed in the following experimental examples, the mixture of shiitake mushroom mycelium culture (LE) and natto bacteria culture (NT) has remarkably superior antihypertensive efficacy to each of the separated shiitake mushroom mycelium culture (LE) and natto bacteria culture (NT).

### Experimental Example 1

### Antihypertensive Effect of Mixture Composition on Non-Genetic Animal Model (Hypertension-Induced Rat Model)

The mixture composition according to an exemplary embodiment of the present disclosure was administered to a non-genetic rat model (hypertensive rats whereby normal rats are artificially treated with materials such as NAME) to accelerate generation of nitrogen monoxide (NO). And then, the researchers observed the status of the rats to ascertain whether their hypertension may be suppressed.

For this purpose, 8-week-old male hypertension-induced SD rats (230-250 g) were fed with free water and standard chow diet (Purina Mills, LLC, St Louis) in a rat-breeding farm where temperature, humidity, and light are adjustable. After one week of adjustment period, the rats were used for the experimentation. 5 rats were allocated to each group.

Here, the hypertension was provided by dissolving L-N-Nitro-L- arginine methyl ester hydrochloride (NAME) (40 mg/kg body weight) into drinking water (30 ml/rat/day). As a positive control group, the hydralazine (50 mg/kg body weight) was dissolved into drinking water (30 ml/rat/day) to be provided. In addition, each of the mixture compositions according to the exemplary embodiments 2-3 (LE, NT, LN1, LN5) was mixed into 20 ml of drinking water in the amount of 10 ml to be provided daily.

After the specimen treatment, the systolic blood pressure (SBP) and the diastolic blood pressure (DBP) of the rats were measured by the Tail-cuff method every week. 5 weeks after the specimen treatment, the rats were anesthetized for each of their organs to be collected. Their blood was collected from the hearts.

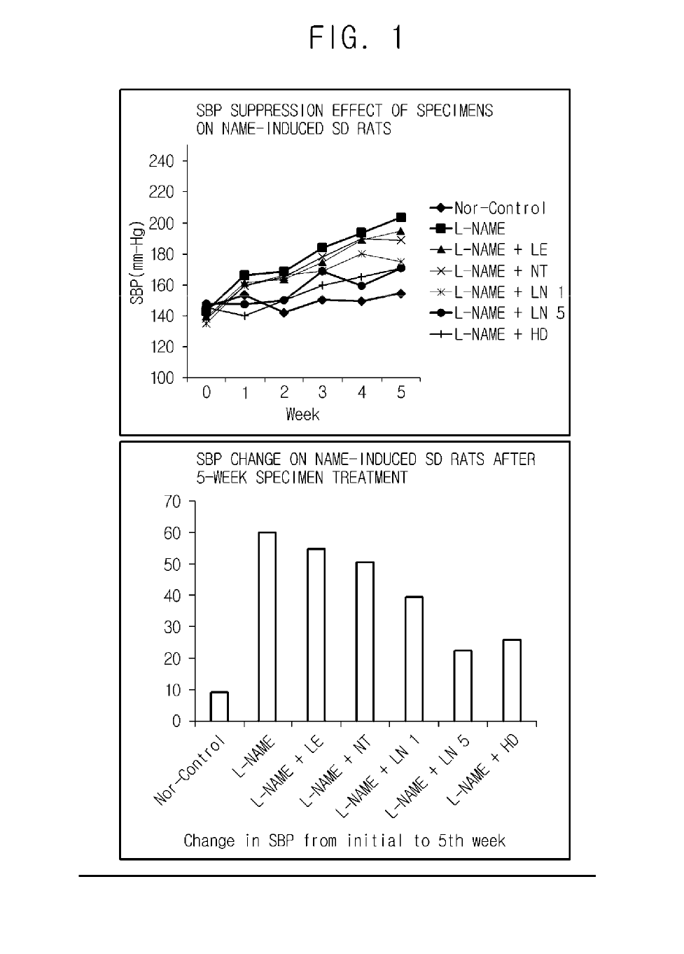

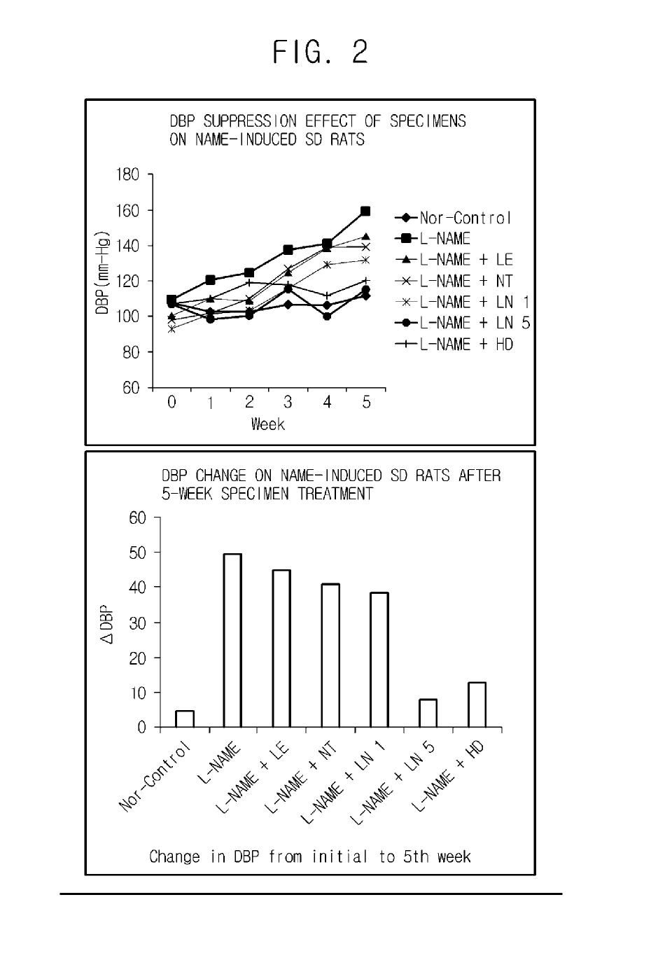

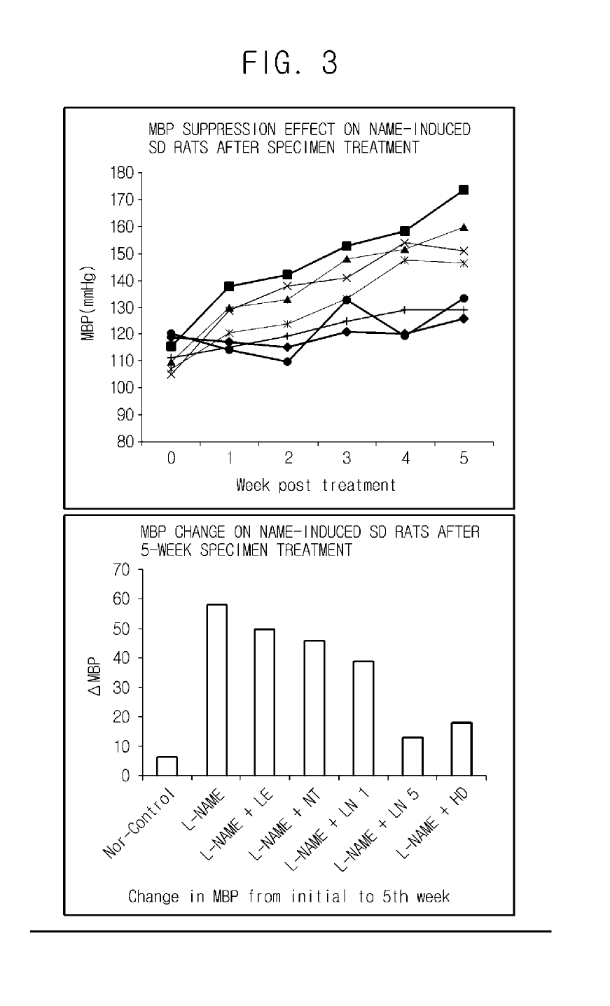

The result is as illustrated in FIGS. 1 to 3: FIG. 1 is a graph illustrating a systolic blood pressure (SBP) drop effect of a mixture of shiitake mushroom mycelium culture and natto bacteria culture on L-N-Nitro-L- arginine methyl ester hydrochloride (NAME)-induced hypertensive rats according to an exemplary embodiment of the present disclosure; FIG. 2 is a graph illustrating a diastolic blood pressure (DBP) drop effect of a mixture of shiitake mushroom mycelium culture and natto bacteria culture on L-N-Nitro-L- arginine methyl ester hydrochloride (NAME)-induced hypertensive rats according to an exemplary embodiment of the present disclosure; and FIG. 3 is a graph illustrating a mean blood pressure (MBP) drop effect of a mixture of shiitake mushroom mycelium culture and natto bacteria culture on L-N-Nitro-L-arginine methyl ester hydrochloride (NAME)-induced hypertensive rats according to an exemplary embodiment of the present disclosure.

In FIGS. 1 to 3, the L-NAME is a hypertension-induced group. The L-NAME-HD is a group treated with hydralazine. The Nor-control is a control group with no treatment.

In general, the nitrogen monoxide (NO) suppresses increasing of the blood pressure by extending the blood vessels. Thus, when activation of NOS enzyme is suppressed, the blood pressure is increasing. NAME has similar structure to L-arginine which is the substrate of NOS. Therefore, NAME suppresses activity of NOS to increase the blood pressure. Therefore, the hydralazine compound treatment group (NAME+hydralazine) shows the effect of suppressing the effect of NAME [that is, NAME is the activity inhibitor of NOS (nitric oxide synthase), and the hydralazine is the accelerant of NOS activity to suppress the blood pressure].

As shown in FIGS. 1 to 3, in case of the NT (natto bacteria culture) and the LE (shiitake mushroom mycelium culture) groups, the blood pressure was decreased after 5 weeks compared to that of the hypertension-induced group (L-NAME). However, the difference is insignificant. On the other hand, in the cases of the mixture composition according to an exemplary embodiment of the present disclosure (LN1, LN5), the blood pressure was significantly decreased compared to the NT (natto bacteria culture) and the LE (shiitake mushroom mycelium culture) groups. Especially, it is known that the specimen LN5 decreased the blood pressure on the similar level to the negative control group (with no treatment) and the positive control group (with hydralazine treatment).

### Experimental Example 2

### Antihypertensive Effect of Mixture Composition on Genetic Animal Model (SHR) by Single Administration

The mixture composition according to an exemplary embodiment of the present disclosure was administered to a genetic rat model [spontaneous hypertensive rat (SHR) whereby their kidney (the kidney takes an important role to adjust the blood pressure) lose the ability to adjust the blood pressure] to increase functionality of the kidney by materials such as ACE enzyme activity inhibitor. The researchers observed the status of the rats to ascertain whether their hypertension may be suppressed.

For this purpose, 8-week-old male SHR (230-250 g) were fed with free water and standard chow diet (Purina Mills, LLC, St Louis) in a cage where temperature, humidity, and light are adjustable. After one week of adjustment period, the rats were used for the experimentation. 8 rats were allocated to each group. And then, each of the mixture compositions according to the exemplary embodiments 2-3 (LE, NT, LN1, LN5) was concentrated and diluted with distilled water with the concentration of 1000 mg per Kg (body weight of the rat) to be orally administered.

As a positive control group, the captopril (CP: 20 mg/Kg) was dissolved into 200 uL distilled water to be orally administered. The systolic blood pressure (SBP) and the diastolic blood pressure (DBP) of the rats were measured by the Tail-cuff method at 1, 3, 6, 12, and 24 hours before and after the specimen treatment.

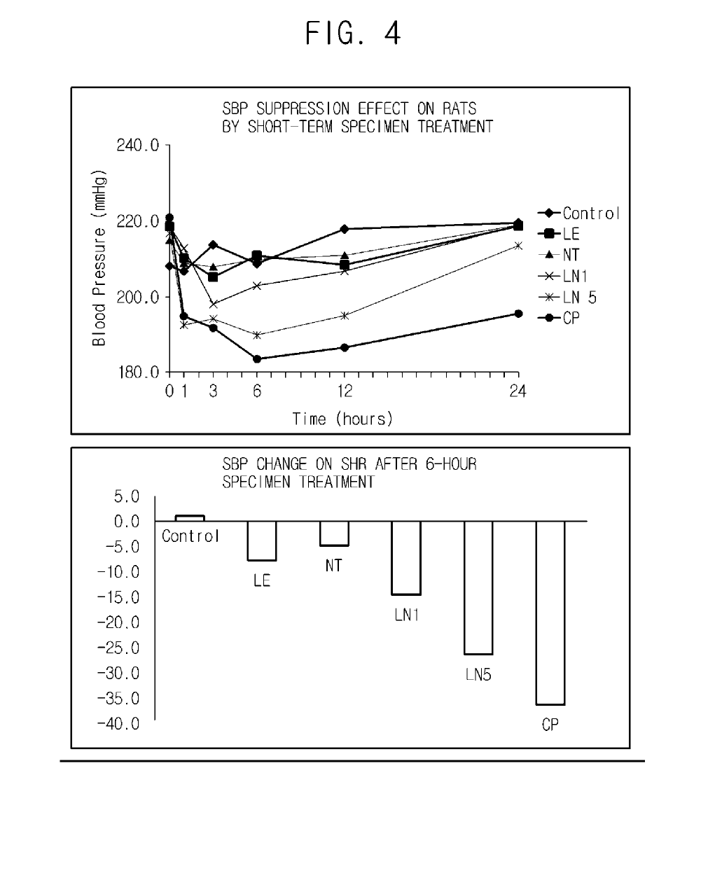

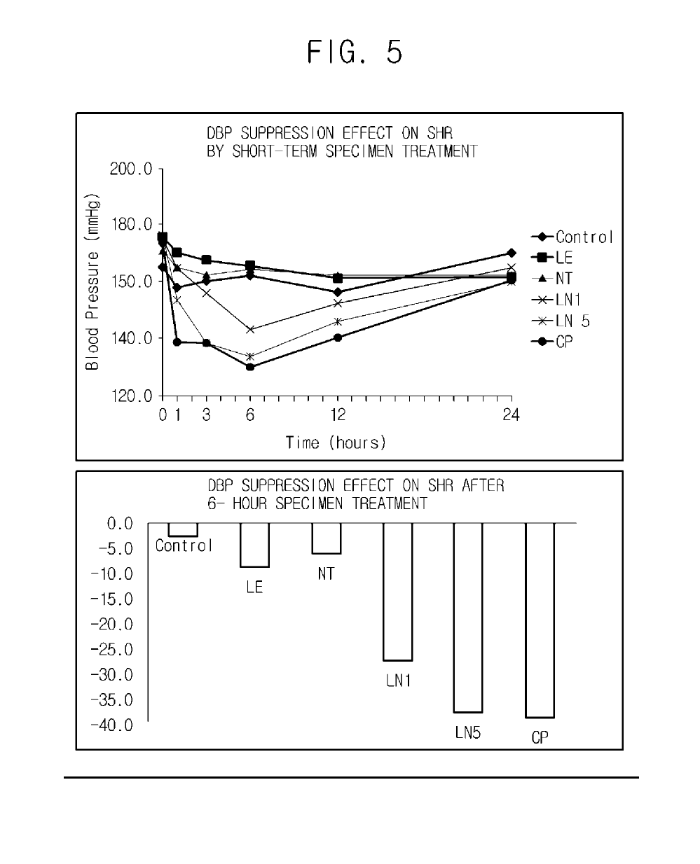

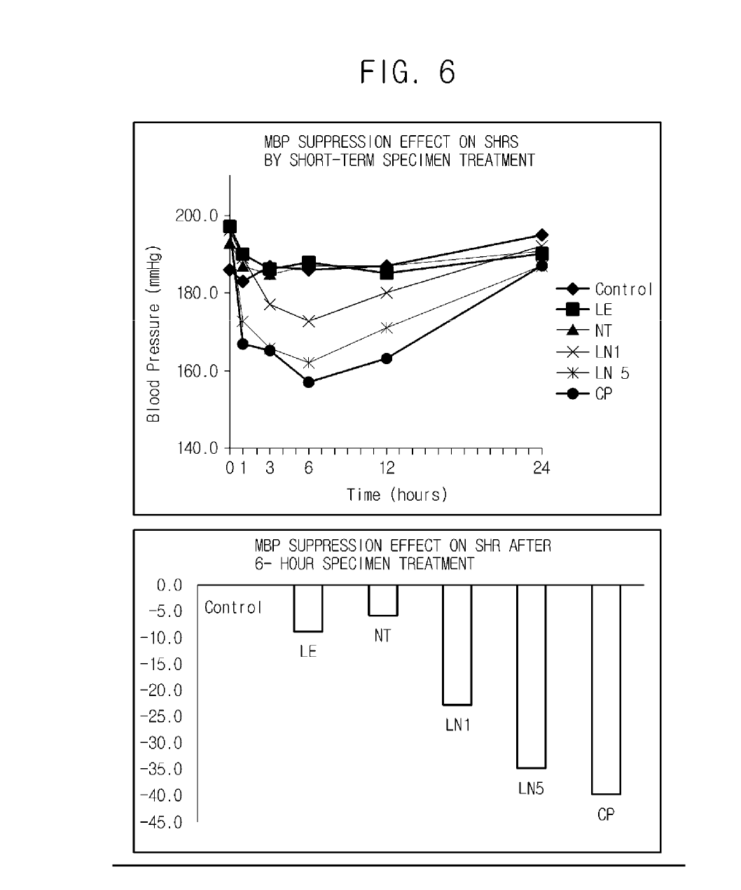

The result is illustrated in FIGS. 4 to 6: FIG. 4 is a graph illustrating a systolic blood pressure (SBP) drop effect of a mixture of shiitake mushroom mycelium culture and natto bacteria culture by short-term treatments on spontaneously hypertensive rat (SHR) according to an exemplary embodiment of the present disclosure; FIG. 5 is a graph illustrating a diastolic blood pressure (DBP) drop effect of a mixture of shiitake mushroom mycelium culture and natto bacteria culture by short-term treatments on spontaneously hypertensive rat (SHR) according to an exemplary embodiment of the present disclosure; and FIG. 6 is a graph illustrating a mean blood pressure (MBP) drop effect of a mixture of shiitake mushroom mycelium culture and natto bacteria culture by short-term treatments on spontaneously hypertensive rat (SHR) according to an exemplary embodiment of the present disclosure.

In FIGS. 4 to 6, the CP is a positive control group with captopril treatment. And the control is a negative control group with no treatment.

As shown in FIGS. 4 to 6, in the result of observing the blood pressure suppression effects on SHR for a short term (24 hours) by high-concentration specimen treatment, the blood pressure suppression effects (SBP, DBP, and MBP) were outstanding when 6 hours were passed after the treatment in totality. Among the specimens, the mixture composition according to an exemplary embodiment of the present disclosure (LN5, LN1) showed the most outstanding effects. Especially, the specimen LN5 decreased the blood pressure on the similar level to the CP which is ACE activity inhibitor.

### Experimental Example 3

### Antihypertensive Effect of Mixture Composition on Genetic Animal Model (SHR) by Long-Term Administration

The short-term administration experiment of the experimental example 2 in the above was performed in a short term using high-concentration specimens. This is to prepare for the sudden increase of blood pressure, and also to experiment the effect of the mixture composition according to an exemplary embodiment of the present disclosure as a medical composition.

On the contrary, here in the experimental example 3, the specimens were administered with low concentration for a long period of 4 weeks to observe the blood pressure suppression effects. This is to experiment the effect of the mixture composition according to an exemplary embodiment of the present disclosure as a health functional food.

Basically, the experimentation was performed by the same method as that of the experimental example 2. However, each of the mixture compositions according to the exemplary embodiments 2-3 (LE, NT, LN1, LN5) was mixed into 20 ml of drinking water in the amount of 10 ml to be provided. As a positive control group, the captopril (CP: 20 mg/Kg) was dissolved into 30 ml drinking water to be orally administered. The systolic blood pressure (SBP), the diastolic blood pressure (DBP), and the heart rate (HR) of the rats were measured by the Tail-cuff method at 1, 2, 3, and 4 weeks before and after the specimen treatment. 4 weeks after the specimen treatment, the rats were anesthetized for each of their lungs to be collected. Their blood was collected from the hearts.

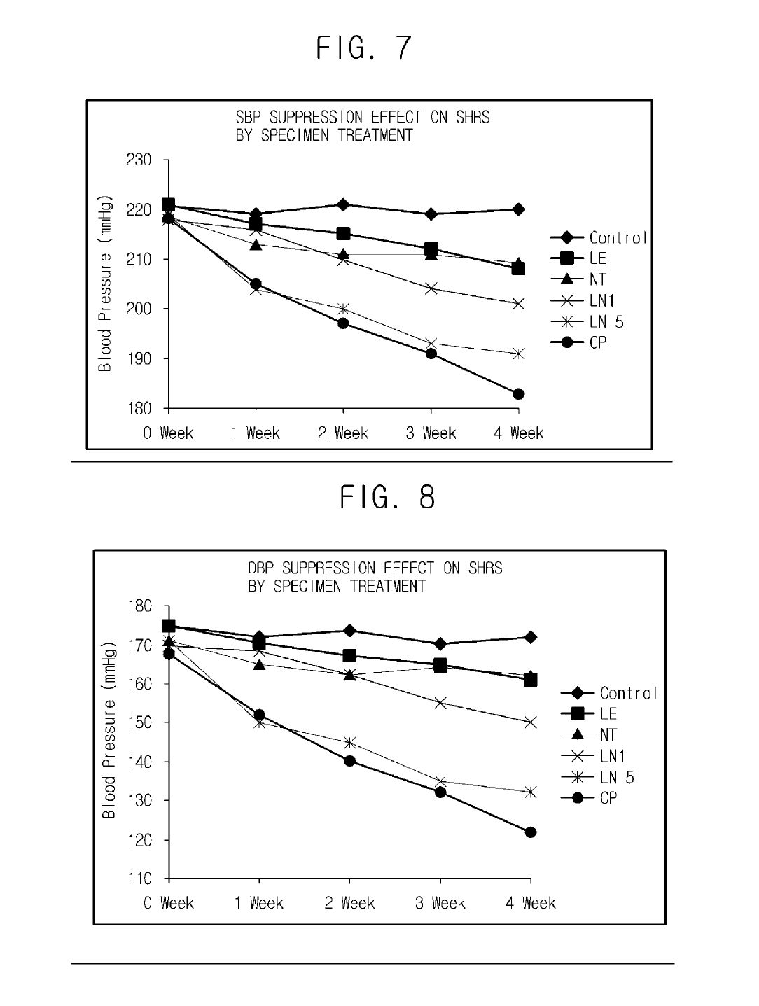

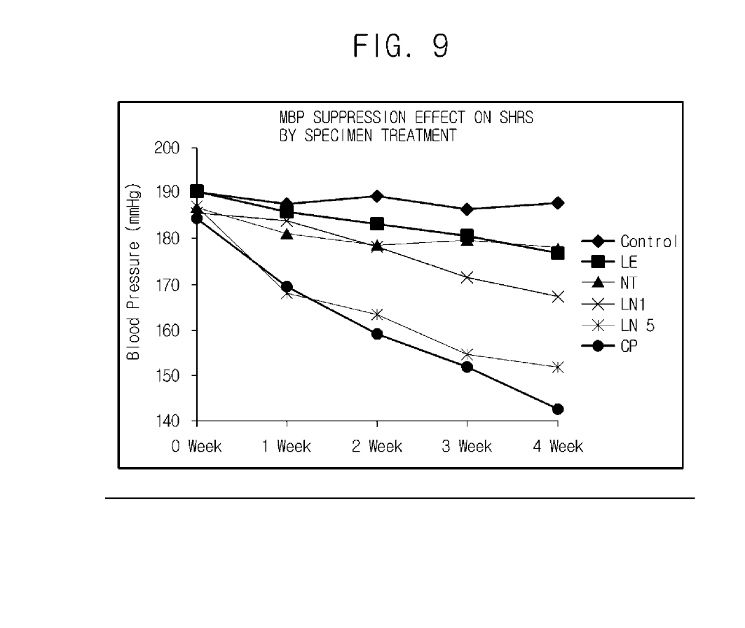

The result is as illustrated in FIGS. 7 to 9: FIG. 7 is a graph illustrating a systolic blood pressure (SBP) drop effect of a mixture of shiitake mushroom mycelium culture and natto bacteria culture by long-term treatments on spontaneously hypertensive rat (SHR) according to an exemplary embodiment of the present disclosure; FIG. 8 is a graph illustrating a diastolic blood pressure (DBP) drop effect of a mixture of shiitake mushroom mycelium culture and natto bacteria culture by long-term treatments on spontaneously hypertensive rat (SHR) according to an exemplary embodiment of the present disclosure; and FIG. 9 is a graph illustrating a mean blood pressure (MBP) drop effect of a mixture of shiitake mushroom mycelium culture and natto bacteria culture by long-term treatments on spontaneously hypertensive rat (SHR) according to an exemplary embodiment of the present disclosure.

In FIGS. 7 to 9, the CP is a positive control group with captopril treatment. And the control is a negative control group with no treatment.

As shown in FIGS. 7 to 9, in the result of observing the blood pressure suppression effects on SHR for a long term (4 weeks) by low-concentration specimen treatment, the blood pressure suppression effects (SBP, DBP, and MBP) were outstanding when 4 weeks were passed after the treatment in totality. Among the specimens, the mixture composition according to an exemplary embodiment of the present disclosure (LN1, LN5) showed the most outstanding effects. Especially, the specimen LN5 decreased the blood pressure on the similar level to the CP which is ACE activity inhibitor.

### Experimental Example 4

### Determining Biomarkers of Mixture Composition Relating to Blood Pressure Adjustment

The biochemical markers relating to the blood pressure were measured, by collecting blood and organs of the rats used in the experimental examples 1 (non-genetic NAME-induced hypertension) and 3 (4-week-treatments on SHR).

This is to discover how the mixture composition according to an exemplary embodiment of the present disclosure decreases the blood pressure.

From the rats of the experimental example 1, NO and eNOS of the blood and the lungs were measured. And from the rats of the experimental example 3, ACE activity and the activity of plasma plasminogen activator inhibitor-1 (PAI-1) of the plasma and the livers were measured. The result is as described in the following TABLE 5 and 6.

The following TABLE 5 is describing NO content and NOS activity of shiitake mushroom mycelium culture and natto bacteria culture mixture in the NAME-induced hypertensive rat model.

TABLE 5

| | | | | | | NOS | | | |
|---|---|---|---|---|---|---|---|---|---|
| | | | | | | | | Aorta | |
| | | NO (nM/ml) | | | | Plasma | | (NO pmle/mg | |
| | Specimen | Plasma | | Lung | | (NO umole/L) | | protein/min) | |
| NO. | name | 0 week | 4 weeks | 0 week | 4 weeks | 0 week | 4 weeks | 0 week | 4 weeks |
| 1 | NAME + LE | 19 | 18 | 11 | 9 | 6.0 | 7.1 | 0.79 | 0.80 |
| 2 | NAME + NE | 21 | 19 | 11 | 8 | 7.0 | 7.5 | 0.82 | 0.88 |
| 3 | NAME + LN1 | 22 | 17 | 10 | 7 | 7.2 | 8.5 | 0.77 | 0.89 |
| 4 | NAME + LN5 | 15 | 13 | 12 | 6 | 7.2 | 8.4 | 0.75 | 0.99 |
| 5 | NAME + | 21 | 20 | 10 | 7 | 6.0 | 8.5 | 0.80 | 1.08 |
| | Hydralizine | | | | | | | | |
| 6 | Control | 20 | 21 | 9 | 11 | 6.5 | 6.0 | 0.73 | 0.76 |
| 7 | NAME | 20 | 11 | 10 | 5 | 6.0 | 2.5 | 0.80 | 0..88 |

As shown in the above TABLE 5, the specimens 3 and 4 suppressed NO generation more efficiently than the other specimens, and also suppressed activity of NOS. The effect was similar to that of the specimen 5, the NO activity accelerant.

In addition, the following TABLE 6 is describing ACE activity and PAI-1 content of shiitake mushroom mycelium culture and natto bacteria culture mixture in SHR.

TABLE 6

| | Speci- | ACE (IC50 nM) | | | | Plasma PAI-1 | |
|---|---|---|---|---|---|---|---|
| | men | Plasma | | Lung | | (pg/ml) | |
| NO. | name | 0 week | 4 weeks | 0 week | 4 weeks | 0 week | 4 weeks |
| 1 | LE | 112 | 90 | 131 | 111 | 121 | 132 |
| 2 | NE | 121 | 91 | 131 | 121 | 115 | 123 |
| 3 | LN1 | 115 | 82 | 125 | 98 | 110 | 132 |
| 4 | LN5 | 109 | 70 | 120 | 80 | 121 | 164 |
| 5 | CP | 111 | 71 | 122 | 81 | 113 | 127 |
| 6 | Control | 123 | 121 | 132 | 121 | 115 | 121 |

As shown in the above TABLE 6, the specimens 3 and 4 suppressed ACE activity (effect of LE) in plasma and lungs efficiently. The effect was similar to that of CP. In addition, the specimens 3 and 4 increased activity of PAI-1 (effect of NT) remarkably superior to the other specimens. According to this, it is determined that LN decreases the blood pressure by suppressing ACE activity and accelerating PAI-1 activity (that is, the mixed effect of LE and NT).

As mentioned in the above, in a non-genetic model, the LN specimen according to an exemplary embodiment of the present disclosure may decrease the blood pressure by accelerating NO generation. And in a genetic model, the LN specimen according to an exemplary embodiment of the present disclosure may suppress the blood pressure by accelerating PAI-1 activity along with suppressing ACE activity.

Although some exemplary embodiments are described and illustrated in the above, it is apparent to the persons skilled in the art that the present disclosure may be variously altered or modified within the limitation of the technical features of fields of the present disclosure provided by the following claims.

1. An antihypertensive composition, the antihypertensive composition comprising shiitake mushroom mycelia and natto bacterial cultures.

2. The antihypertensive composition of claim 1, wherein
the shiitake mushroom mycelium culture is obtained by culturing shiitake mushroom (Lentinus edodes) strain, and
the natto bacteria culture is obtained by culturing natto bacterium (Bacillus subtilis natto).

3. The antihypertensive composition of claim 1, wherein the ratio of
the shiitake mushroom mycelium culture and the natto bacteria culture are in a weight ratio of 99˜95:1˜5, respectively.

4. The antihypertensive composition of claim 1, wherein
the shiitake mushroom mycelium culture is obtained by culturing shiitake mushroom (Lentinus edodes) strain in a medium to which adenine or ribose, or both are added.

5. The antihypertensive composition of claim 1, wherein
the shiitake mushroom mycelium culture is obtained by culturing shiitake mushroom (Lentinus edodes) strain in a medium containing food additive or yeast extract containing adenine and ribose.

6. The antihypertensive composition of claim 1, wherein
the shiitake mushroom mycelium culture is obtained by culturing mycelia, obtained by culturing shiitake mushroom (Lentinus edodes) strain, in a medium in which adenine or ribose or both are naturally present.

7. The antihypertensive composition of claim 4, wherein
the medium contains the adenine and the ribose in an amount of 0.1˜0.5 weight % of a total weight of the medium.

8. A hypertension therapeutic or preventive pharmaceutical composition, comprising the antihypertensive composition of claim 1.

9. An antihypertensive health food, comprising the antihypertensive composition of claim 1.

10. A method for increasing EA content in shiitake mushroom mycelium culture, comprising culturing shiitake mushroom (Lentinus edodes) strain in a medium containing adenine and ribose.

11. A method of claim 10, wherein
the medium contains each of the adenine and the ribose in an amount of 0.1˜0.5 weight % of a total weight of the medium.
